(19) United States
(12) Patent Application Publication
(10) Pub. No.: US 2017/0007749 A1
(43) Pub. Date: Jan. 12, 2017

(54) THORAX DRAINAGE DEVICE

(71) Applicant: MEDELA HOLDING AG, Baar (CH)

(72) Inventors: Martin Walti, Zurich (CH); Hilmar Ehlert, Hergiswil (CH)

(73) Assignee: MEDELA HOLDING AG, Baar (CH)

(21) Appl. No.: 15/112,670

(22) PCT Filed: Jan. 27, 2015
(86) PCT No.: PCT/EP2015/051557

§ 371 Date: Jul. 19, 2016

(30) Foreign Application Priority Data
Jan. 30, 2014 (CH) 0123/14

(51) Int. Cl. A61M 1/00

(52) U.S. Cl. CPC A61M 1/0035; A61M 1/0001; A61M 2205/3344; A61M 2230/42; A61M 2210/101

(57) ABSTRACT

A thorax drainage device for aspirating fluids from a pleural cavity of a patient using underpressure has a fluid collection container for collecting the aspirated fluids and a drainage tube for connecting the fluid collection container to the pleural cavity of the patient. The fluid collection container is connectable to a vacuum source, in order to generate an underpressure in the fluid collection container. The thorax drainage device has an adjustable mechanism for attenuating pressure differences during the respiration of the patient, this mechanism being adjustable independently of a suction capacity of the vacuum source. This device permits a gradual expansion of the lung without risk of injury and thus prepares the lung for the completion of the drainage.

### CROSS-REFERENCE TO RELATED APPLICATION

The present application is the US national phase of International Patent Application No. PCT/EP2015/051557, filed Jan. 27, 2015, which application claims priority to Switzerland Application No. CH 0123/14, filed Jan. 30, 2014. The priority application, CH 0123/14, is hereby incorporated by reference.

### TECHNICAL FIELD

The present invention relates to a thorax drainage device, a chamber for use in such a device, and a method for thorax drainage.

### PRIOR ART

Thorax drainage serves to convey blood, secretions or air from the pleural cavity. The pleural cavity is the space between the visceral pleura and the parietal pleura. The pleural cavity is filled with a serous liquid and is physiologically at a relative underpressure with respect to the outside air, which underpressure rises further during inhalation. Thus, during inhalation, the lung has to follow the active expansion of thoracic wall muscles and the diaphragm. If the relative underpressure in the pleural cavity is cancelled, e.g. during an operation or in an accident, the lung no longer follows the expanding thoracic cage during inhalation. The defect, which leads to air entering the pleural cavity, is generally called an air fistula.

Thorax drainage serves to maintain or restore the physiological underpressure. The thoracic cage and the pleura are opened through an intercostal space, a drainage tube is inserted and, finally, controlled suction is applied in order to drain the pleural cavity. Drainage is most commonly used in connection with operations in which the thoracic cage has to be opened.

Various thorax drainage devices are known in the prior art. As is shown in FIG. 1, they usually have a suction pump 1, operated by an electric motor, or a wall vacuum, these being connected via a suction line 2 to a fluid collection container 3 and generating an underpressure in this fluid collection container 3. A drainage tube 4 leads from the fluid collection container 3 to the pleural cavity P in order to aspirate fluid from the pleural cavity P into the fluid collection container. In FIG. 1, the lung is designated by reference sign L.

U.S. Pat. No. 5,738,656 discloses a drainage device with a drainage line and with an auxiliary line by means of which the drainage tube is flushed and the suction pressure can be controlled. WO 2009/005424 describes a drainage device in which the underpressure in the fluid collection container is controlled by means of a sensor, wherein the sensor is arranged in the suction line leading to the suction pump.

WO 2012/162848 proposes an adaptive algorithm for thorax drainage therapy, wherein a suitable size parameter for the air fistula is determined and the vacuum generated by the suction pump is regulated according to this size parameter.

U.S. Pat. No. 6,261,276 discloses a manually operated thorax drainage device with a bellows-shaped fluid collection container. This bellows serves as a vacuum pump and at the same time as an indicator for the underpressure generated in the fluid collection container.

U.S. Pat. No. 8,177,763 discloses a drainage device with a vacuum chamber, which is connected to a vacuum source, and with a fluid collection container, which is connected fluidically to the vacuum chamber via a hydrophobic membrane.

When the drainage tube is removed from the pleural cavity upon completion of the treatment, there is a danger of overexpansion of the lung, which can lead to a pneumothorax. The reason for this is a sudden increase in the pressure amplitude upon deep inhalation, i.e. a greatly increased underpressure in the pleural cavity, which also overexpands the lung.

### DISCLOSURE OF THE INVENTION

It is therefore an object of the invention to minimize the risk of excessive overexpansion of the lung upon completion of the thorax drainage.

This object is achieved by a thorax drainage device having the features of Patent claim 1, a chamber having the features of Patent claim 16 and for use in such a thorax drainage device, and a thorax drainage method having the features of Patent claim 17.

The thorax drainage device according to the invention for aspirating fluids from a pleural cavity of a patient by means of underpressure has a fluid collection container for collecting the aspirated fluids and a drainage tube for connecting the fluid collection container to the pleural cavity of the patient. The fluid collection container is connectable to a vacuum source, in order to generate an underpressure in the fluid collection container. The thorax drainage device has an adjustable mechanism for attenuating pressure differences during the respiration of the patient, this mechanism being adjustable independently of a suction capacity of the vacuum source.

It is thus possible, with drainage parameters otherwise remaining unchanged, to accustom the patient to the completion of the drainage. It is thus possible, during the thorax drainage itself, to permit ever greater pressure differences during respiration and to train the lung to cope with greater expansion without damage. The risk of overexpansion of the lung, with subsequent pneumothorax, upon completion of the drainage is thus greatly reduced.

Preferably, the mechanism for attenuating pressure differences is a mechanism for adjusting a return flow of air to the pleural cavity. The hardness or flexibility of the thorax system can thereby be adjusted. The expansion of the lung, hence the attenuation of the pressure differences in the pleural cavity, is dependent on the possible quantity of air flowing back into the pleural cavity.

The adjustment of the return flow of air can be effected manually or automatically. In one embodiment, the automatic adjustment can be regulated automatically according to a sensor value. That is to say, the adjustment is not left the same for a long period of time, e.g. several hours or days. Instead, it is constantly regulated, in order to also attenuate sudden increases in pressure difference, e.g. when the patient overexerts himself or unintentionally breathes in too deeply. The sensor value is preferably a pressure detected in the container, in the drainage tube or in the pleural cavity.

Preferably, the mechanism for attenuating pressure differences is arranged between secretion collection container and suction source or in the housing of the suction source or in or on the secretion collection container or in or on the drainage tube.

In a preferred embodiment, the mechanism for attenuating pressure differences has a chamber, of which the stiffness is adjustable. Adjustability of the stiffness here means the stiffness of the walls, the change of the volume available for the return of air, and also the delivery of external air into the chamber. This is explained below on the basis of a number of preferred embodiments.

In the embodiments described below, the mechanism for attenuating pressure differences has a chamber with an interior and with an opening that leads to the patient.

In one embodiment, the chamber is formed by stiff walls, with the exception of a flexible membrane let into a wall of the chamber, wherein the flexibility of the membrane is adjustable. The membrane can be spring-loaded, which avoids too great an expansion of the membrane.

In a further embodiment, the chamber is formed by stiff walls, with the exception of a spring-loaded piston which forms part of a wall, and of which the position relative to the interior is adjustable.

In another embodiment, the chamber is formed by stiff walls, wherein an insert container is arranged in the chamber, which insert container can be filled from the outside with a non-compressible fluid in order to limit the volume of the interior adjustably.

In a further embodiment, the chamber is formed by stiff walls, with the exception of a flexible bellows which forms part of a wall and which has an interior open towards the interior of the chamber, wherein the volume of the interior of the bellows is adjustable.

In another embodiment, a first chamber with an interior and with an opening leading to the patient is present, wherein the first chamber is formed by stiff walls, wherein one wall has a closable first air exchange opening. The latter serves for connection to a second chamber, which is closed except for a second air exchange opening, wherein the first chamber can be connected to the second chamber for air communication via the two air exchange openings.

In a further embodiment, the chamber is formed by stiff walls, wherein the chamber has a filling opening, which is independent of any suction opening connected to the suction source and through which air can be blown or pumped into the chamber for the purpose of adjusting the attenuation of the respiration.

In another embodiment, the chamber is formed by stiff walls, wherein the chamber has a valve which leads to the outside and which opens outwards according to a detected underpressure.

Preferably, the chamber or the first chamber is formed by the fluid collection container. It is alternatively arranged in or on the fluid collection container. Alternatively or in addition, it can also be connected by a branch line to the drainage tube or can be arranged between suction source and fluid collection container.

Further embodiments are set forth in the dependent claims.

### BRIEF DESCRIPTION OF THE DRAWINGS

Preferred embodiments of the invention are described below with reference to the drawings, which serve only for illustration and are not to be interpreted as limiting the invention. In the drawings:

FIG. 1 shows a schematic view of a lung, with a thorax drainage device according to the prior art connected thereto;

FIG. 2a shows a schematic view of a lung during exhalation in existing thorax drainage;

FIG. 2b shows the change of the underpressure in the pleural cavity during the exhalation according to FIG. 2a;

FIG. 3a shows a schematic view of a lung during inhalation in existing thorax drainage;

FIG. 3b shows the change of the underpressure in the pleural cavity during the inhalation according to FIG. 3a;

FIG. 4a shows a schematic view of a lung during exhalation without thorax drainage;

FIG. 4b shows the change of the underpressure in the pleural cavity during the exhalation according to FIG. 4a;

FIG. 5a shows a schematic view of a lung during inhalation in existing thorax drainage;

FIG. 5b shows the change of the underpressure in the pleural cavity during the inhalation according to FIG. 5a;

FIG. 6a shows a schematic view of a lung during thorax drainage according to the prior art;

FIG. 6b shows a schematic view of the pressure in the pleural cavity during the thorax drainage according to FIG. 6a as a function of time;

FIG. 7a shows a schematic view of the lung according to FIG. 6a after completion of the thorax drainage according to the prior art;

FIG. 7b shows a schematic view of the pressure in the pleural cavity after completion of the thorax drainage according to FIG. 7a as a function of time;

FIG. 8 shows a schematic view of a lung and, connected thereto, a thorax drainage device according to the invention in a first embodiment;

FIG. 9a shows a schematic view of a lung during thorax drainage with a fluid collection container according to the invention;

FIG. 9b shows a schematic view of the pressure in the pleural cavity during the thorax drainage according to the invention as per FIG. 9a, as a function of time;

FIG. 10a shows a schematic view of a lung after completion of thorax drainage with a fluid collection container according to the invention;

FIG. 10b shows a schematic view of the pressure in the pleural cavity after completion of the thorax drainage according to the invention as per FIG. 10a, as a function of time;

FIG. 11 shows a schematic view of a fluid collection container according to the invention in a first embodiment;

FIG. 12 shows a schematic view of a fluid collection container according to the invention in a second embodiment;

FIG. 13 shows a schematic view of a fluid collection container according to the invention in a third embodiment;

FIG. 14 shows a schematic view of a fluid collection container according to the invention in a fourth embodiment;

FIG. 15 shows a schematic view of a fluid collection container according to the invention in a fifth embodiment;

FIG. 16 shows a schematic view of a fluid collection container according to the invention in a sixth embodiment;

FIG. 17 shows a schematic view of a fluid collection container according to the invention in a seventh embodiment;

FIG. 18 shows a schematic view of a fluid collection container according to the invention in an eighth embodiment;

FIG. 19 shows a schematic view of a fluid collection container according to the invention in a ninth embodiment;

FIG. 20 shows a schematic view of the pressure in the pleural cavity during the thorax drainage according to the invention using one of the fluid collection containers according to FIGS. 18 and 19, as a function of time;

FIG. 21 shows a schematic view of a fluid collection container according to the invention in a tenth embodiment;

FIG. 22 shows a schematic view of the pressure in the pleural cavity during the thorax drainage according to the invention using the fluid collection container according to FIG. 21, as a function of time;

FIG. 23a shows a schematic view of a lung and, connected thereto, a thorax drainage device according to the invention in a second embodiment;

FIG. 23b shows an alternative embodiment to FIG. 23a;

FIG. 24a shows a schematic view of a lung and, connected thereto, a thorax drainage device according to the invention in a third embodiment;

FIG. 24b shows a more concrete view of a variant of the embodiment according to FIG. 24a.

### DESCRIPTION OF PREFERRED EMBODIMENTS

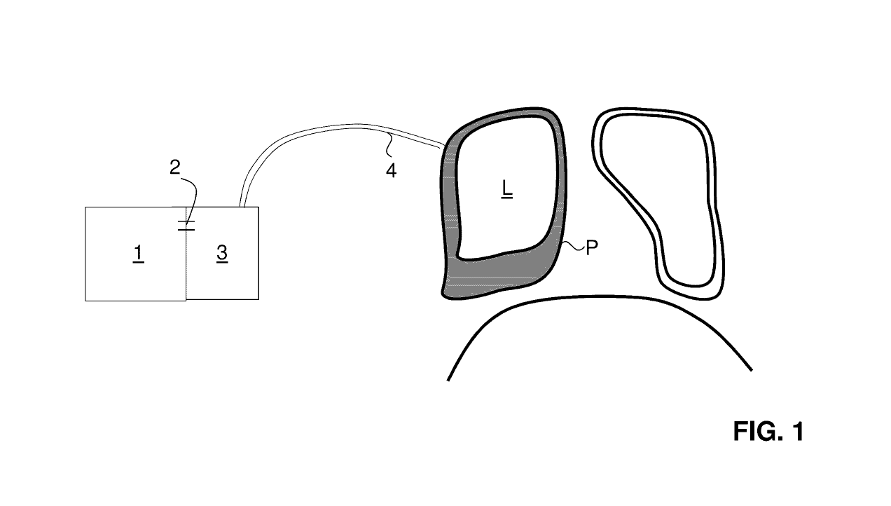

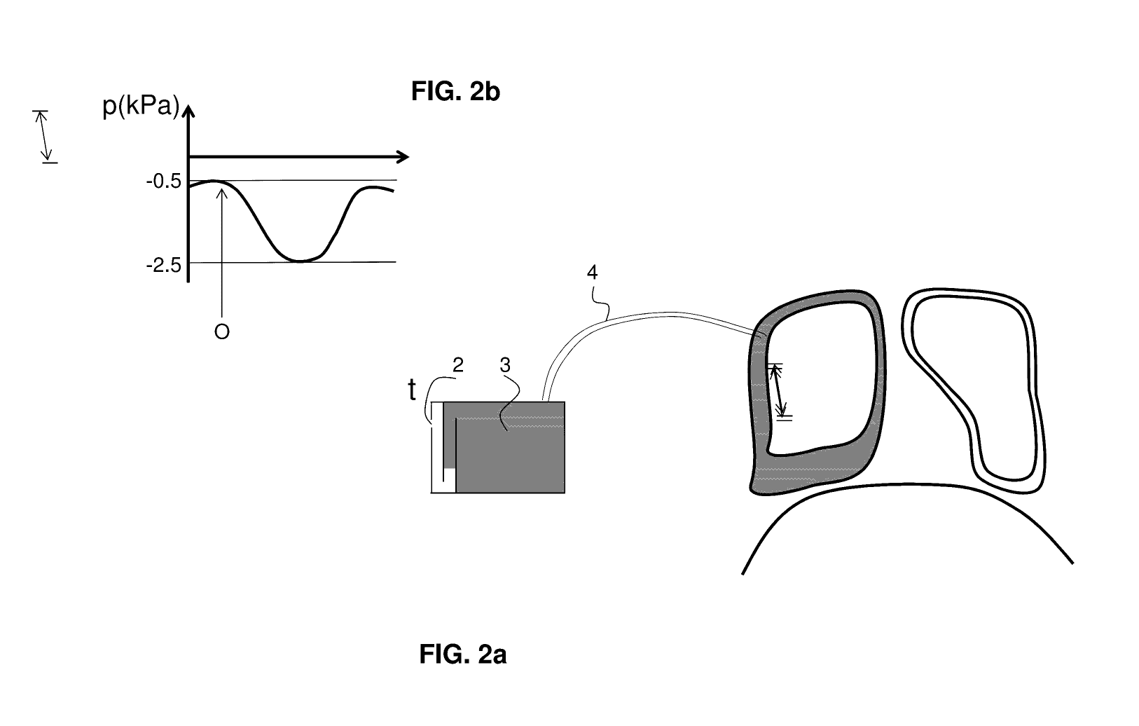

As has already been mentioned above, FIG. 1 shows a lung during thorax drainage. FIG. 2a shows the situation during exhalation. For simplicity, in this figure, and in the subsequent figures, only the drainage tube 4 and the fluid collection container 3 are shown, not the vacuum pump. However, the latter is of course connected to the fluid collection container 3 via the suction line (here shown only with a simple opening 2) during the drainage.

During exhalation, the lung L decreases in size, as is shown schematically by the double arrow in FIG. 2a. The double arrow visualizes the expansion of the lung. The absolute pressure in the pleural cavity drops, i.e. the relative pressure difference in relation to the atmospheric pressure becomes smaller. This is shown in FIG. 2b by the arrow O. During exhalation, the underpressure prevailing in the pleural cavity rises in the direction of atmospheric pressure. In this example, it reaches −0.5 kPa.

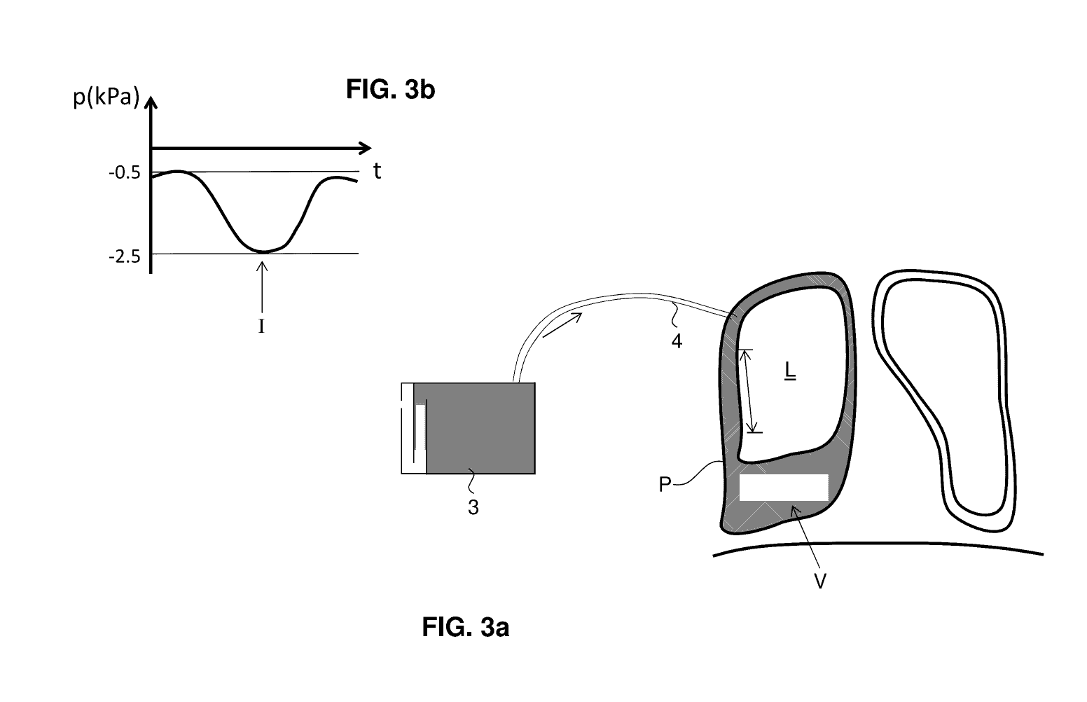

If the patient now inhales during the thorax drainage, the lung L expands. This is shown in FIG. 3a. The volume in the pleural cavity also becomes greater and, on account of the then greater pressure difference in relation to the atmospheric pressure, i.e. on account of the greater absolute underpressure value, it draws air out of the fluid collection container 3 into the pleural cavity. This is shown in FIG. 3a by the rectangular bar and the reference sign V. The negative pressure value during inhalation is indicated by an arrow I in FIG. 3b. In this example, it amounts to −2.5 kPa.

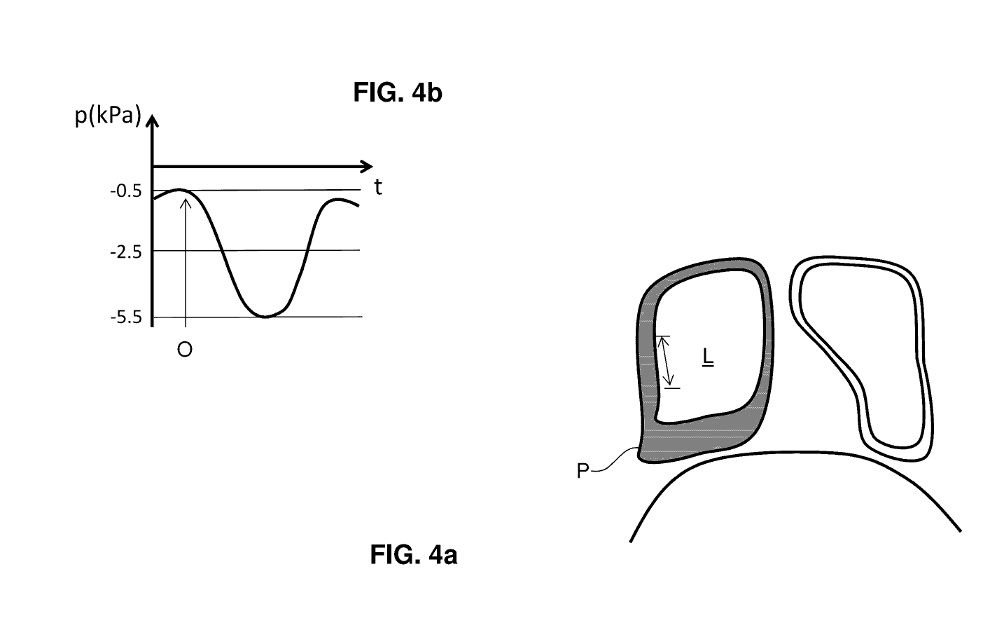

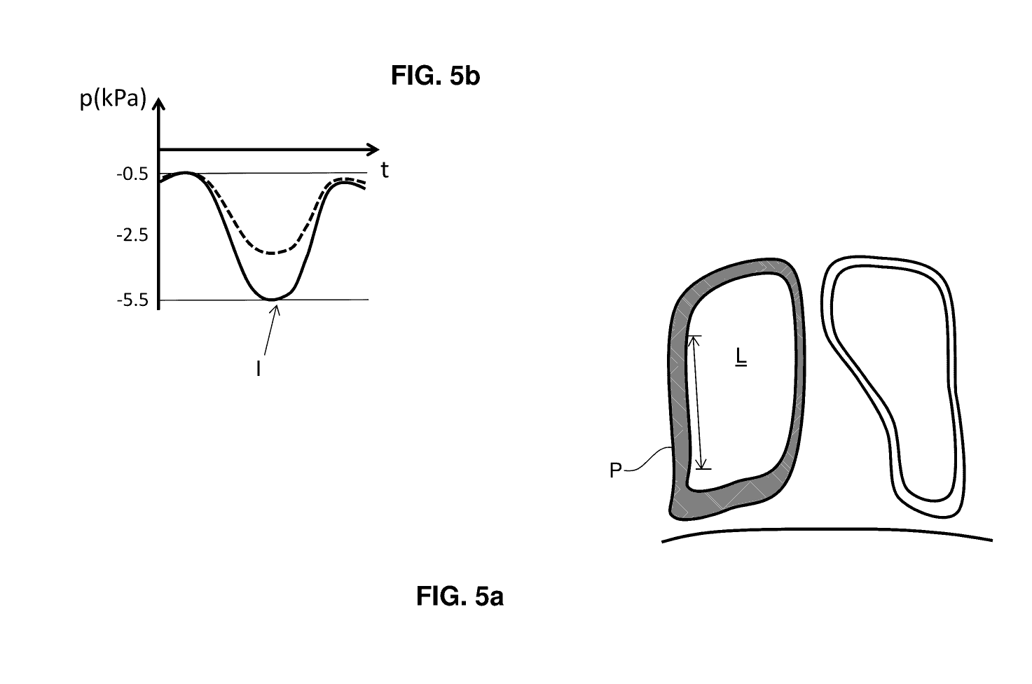

If the drainage tube 4 is clamped, the suction pump is switched off or the entire drainage device removed, the lung once again forms an autonomous system with the pleural cavity, as is shown in FIG. 4a. In FIG. 4b, the arrow O again indicates the pressure value during exhalation. In this example, the value is unchanged at −0.5 kPa. FIG. 5a shows the situation during inhalation without connection to the thorax drainage. Since no air can be drawn from the container into the pleural cavity, the absolute underpressure value in the pleural cavity P rises more strongly. In this example to −5.5 kPa. The dashed line in FIG. 5b shows the expansion during the thorax drainage. Thus, the lung L is able to expand more strongly without the thorax drainage. There is a danger of overexpansion of the lung and therefore of a pneumothorax.

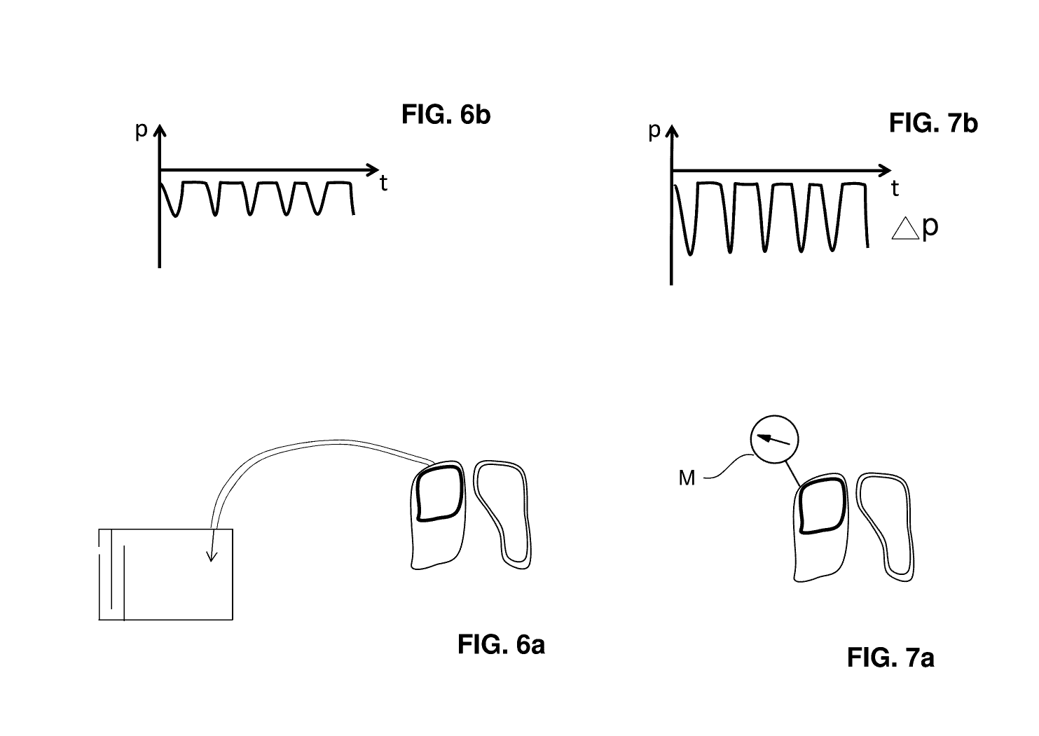

FIGS. 6a and 6b again show the situation during the thorax drainage, while FIGS. 7a and 7b show the situation after completion of the thorax drainage. FIG. 7a shows a manometer M, with which the pressure in the pleural cavity is measured. Δp here designates the pressure difference between inhalation and exhalation. In this view and similar ones, p denotes the pressure in the pleural cavity and t denotes the time.

As can be seen from a comparison of FIGS. 6b and 7b, the increase in the pressure difference after completion or interruption of the drainage occurs abruptly and directly.

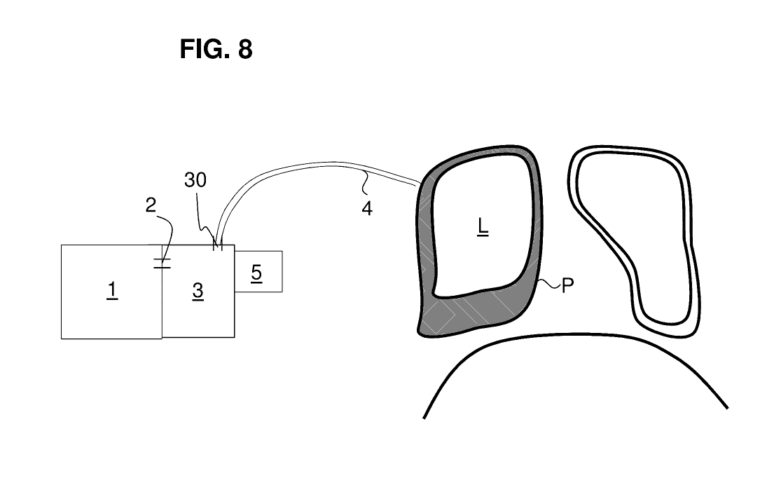

This situation is now intended to be avoided with the thorax drainage device according to the invention. FIG. 8 thus shows a thorax drainage device according to the invention in a first embodiment. This device likewise has a suction mechanism, preferably a suction or vacuum pump 1, which is connected to a fluid collection container 3 via a suction line. From the fluid collection container 3, a drainage tube 4 leads to the pleural cavity P of a patient. Instead of a vacuum pump 1 driven by motor, the fluid collection container 3 can also be attached to a vacuum system within the hospital.

The fluid collection container 3 is stiff. It can be composed of one or more chambers. The at least one chamber can be provided with ribs in order to limit the sloshing around of the aspirated liquid. The fluid collection container 3 has a drainage opening 30 for connection to the drainage tube 4. It also has a suction opening 2 for connection to the suction pump 1. The suction opening 2 is preferably provided with a nonreturn valve and/or a bacteria filter, in order to protect the suction pump 1 from contamination. Containers of this kind are well known in the prior art. The fluid collection container 3 according to the invention can also be smaller than is shown in the prior art.

According to the invention, this fluid collection container 3, which is inherently stiff and of unchangeable internal volume, is provided with a mechanism 5 by which the hardness of the fluid collection container 3 can be adjusted. The system is soft shortly after the operation or at the start of the drainage, and it becomes ever harder and stiffer towards the end of the drainage, such that the lung is able to accustom itself to greater expansions.

The mechanism comprises an inherently closed chamber with an opening that leads to the patient.

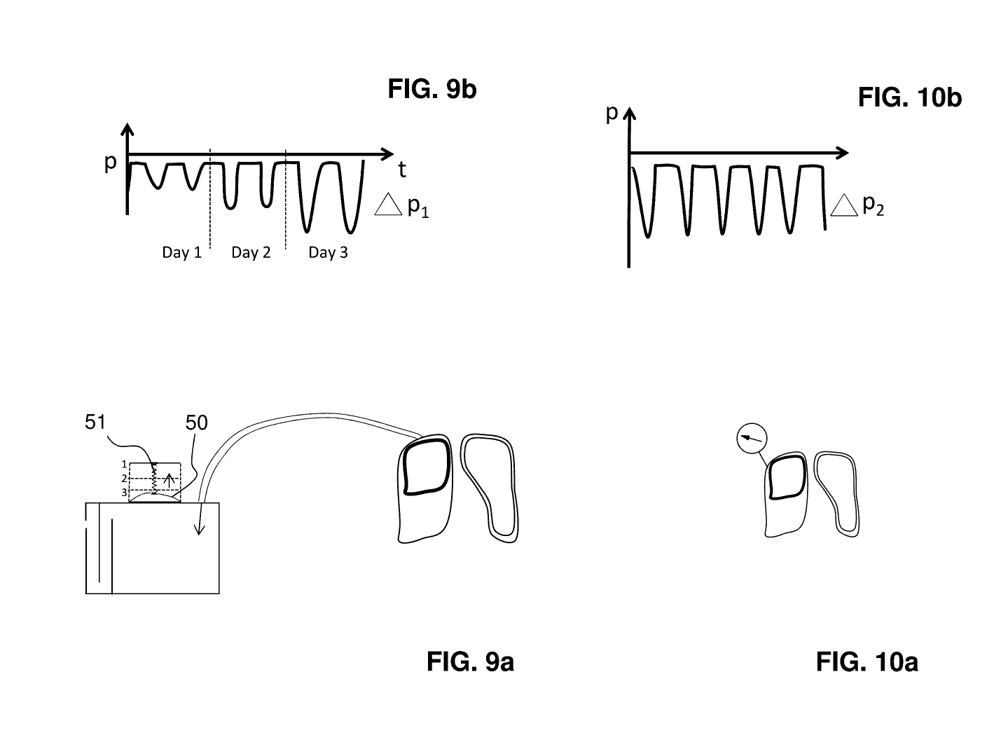

In the embodiment according to FIG. 9a, this mechanism 5 has a membrane 50, which forms part of the outer wall of the fluid collection container 3. The fluid collection container thus forms the abovementioned chamber. This membrane 50 is fluid-tight, in particular airtight. It can be expanded by means of a spring 51, as a result of which its hardness, i.e. its inherent resilience, can be adjusted. Three positions 1, 2, 3 of the spring 51, and therefore of the membrane 50, are shown schematically in FIG. 9a. FIG. 9b shows how the pressure profile in the pleural cavity changes depending on this spring setting. If the spring 51 is in position 3 on day 1, the membrane 50 is barely expanded and very soft. As the pressure difference increases, the fluid collection container 3 changes the volume by virtue of the flexible membrane 50, such that enough air can get into the pleural cavity P and overexpansion of the lung L is prevented.

On the second day, the membrane 50 is made slightly stiffer by being tensioned to a greater extent, for example as far as position 2. The drainage system as a whole becomes harder and stiffer as a result, since the change in volume of the fluid collection container 3 is limited. During inhalation, less air now passes from the fluid collection container 3 into the pleural cavity P. The underpressure in the pleural cavity P can increase. This can be seen in the area designated “Day 2” in FIG. 9b. The lung L can thus expand slightly more. On the third day, the membrane 50 is tensioned and stiffened to a still greater extent, by means of the spring 51 being brought to position 1 according to FIG. 9a. The absolute underpressure value in the pleural cavity P can increase still further, as can be seen in the area “Day 3” in FIG. 9b. Thus, the pressure conditions in the pleural cavity P and the expansion of the lung L can be adapted successively to a state prevailing after completion of the thorax drainage, without abrupt changes in the pressure difference. The situation after completion of the thorax drainage is shown in FIGS. 10a and 10b. As can be seen, Δp₂ is equal to or approximately equal to Δp₁.

According to the invention, the stress on the lung L is thus gradually increased until the drainage is removed. As has been described in this example, the increase can take place daily. However, it can also occur at different time intervals and/or can be interrupted by phases in which the stress is reduced. The medical staff providing the treatment will decide this according to the improvement in the state of health of the individual patient. According to the invention, it is possible to avoid a sudden overexpansion of the lung L after completion and removal of the drainage.

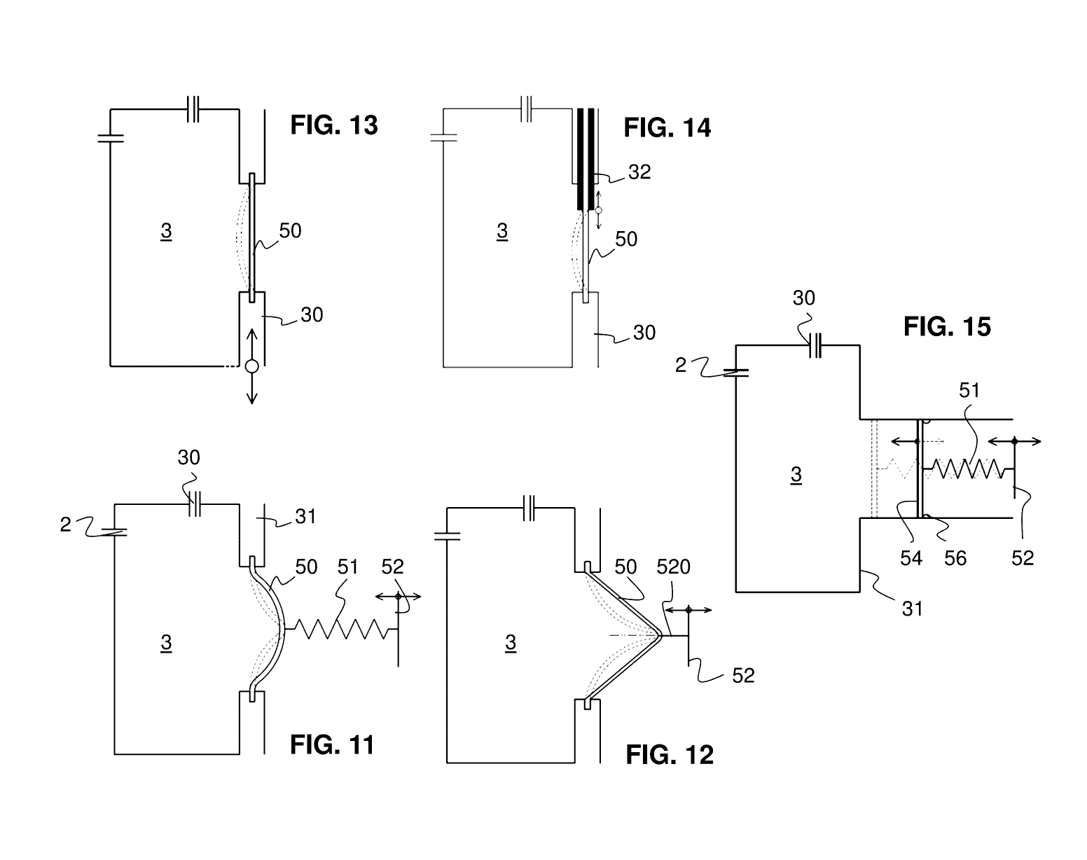

FIG. 11 shows said first embodiment of the fluid collection container 3 according to the invention. Reference number 30 designates the opening for connection to the drainage tube 4, reference number 2 designates the suction opening for connection to the suction pump 1 or the suction line. The membrane 50 is secured in a wall 31 of the otherwise stiff fluid collection container 3, which is designed with an unchangeable internal volume. The membrane 50 can be rectangular, triangular, round, oval or of another shape. It is fluid-tight. It is preferably made of silicone.

Here, the membrane 50 is held and fixed along its outer circumference in the wall 31. For example, it can be adhesively bonded or welded to the wall 31 or can be produced in one piece with the latter by multiple injection moulding.

The spring 51 is preferably connected rigidly to the membrane 50 and is adjustable via a movable anchor 52. The anchor 52 can be fixed in its position relative to the container 3 and is movable relative to the surface of the membrane 50. This is shown in the figure by the double arrow. This also applies to the following examples, which have an anchor or another fixing means.

The anchor 52 can be designed as a slide or knob, for example, or can be connected to an operating element of this kind. It is, for example, a part of an add-on body arranged on the container. An add-on body of this kind is provided with reference number 5 in FIG. 8.

The membrane 50 indicated by a dashed line in FIG. 11 shows the position of the membrane during inhalation, while the membrane 50 indicated by solid lines shows the position during exhalation.

FIG. 12 shows a second embodiment. Here, the membrane 50 is connected by a rigid connecting rod 520 to the anchor 52, which is movable in a direction perpendicular to the membrane surface. Here too, the membrane 50 can be fixed in different expanded positions in order to adjust its resilience and hardness or flexibility. The farther the membrane 50 is drawn away from the container 3 and stretched, the harder the overall system. Once again, the membrane indicated by dashed lines shows its position during inhalation, while the membrane 50 indicated by solid lines shows the position during exhalation.

FIG. 13 shows a third embodiment. The membrane 50 here is adjustable parallel to its surface, i.e. it is stretched or relaxed parallel to its surface. This is shown by the double arrow. This too can be permitted via actuating means, for example a slide or a knob. The same applies here, namely that the more the membrane is stretched, the harder or stiffer is the overall system. The membrane indicated by dashed lines once again shows the situation during inhalation.

In the embodiment according to FIG. 14, a securing element 32 with which the membrane 50 is held in the wall 31 of the container 3 is movable, such that the membrane 50 is stretched to differing extents. As is shown here, the securing element 32 can be designed as a slide or carriage. It can also open and close in the manner of a diaphragm, for example. Otherwise, the same applies here as in the embodiment according to FIG. 13.

In the embodiment according to FIG. 15, part of a wall 31 of the container 3 is rigid but movable. This part forms a piston 54, which is held in a piston housing 55 open to the atmosphere. The piston 54 is sealed off from the outside. Here, for example, a sealing ring 56 is arranged on the outside of the piston 54. This piston 54 is connected by the spring 51 to an adjustable anchor 52. The mobility of the piston 54, and therefore the hardness or flexibility of the container 3, can again be adjusted by the positioning of the anchor 52. The spring 51 here permits the flexibility of the container 3 during inhalation and exhalation. That is to say, the piston 54 moves in the direction of the interior of the container 3 when, during inhalation, the suction force of the vacuum in the interior is greater than the spring force. The position of the anchor 52 influences the hardness of the system.

The embodiments described thus far can be arranged on the container 3. They can also be formed in a separate intermediate container between container 3 and drainage tube 4 or between suction pump 1 and container 3.

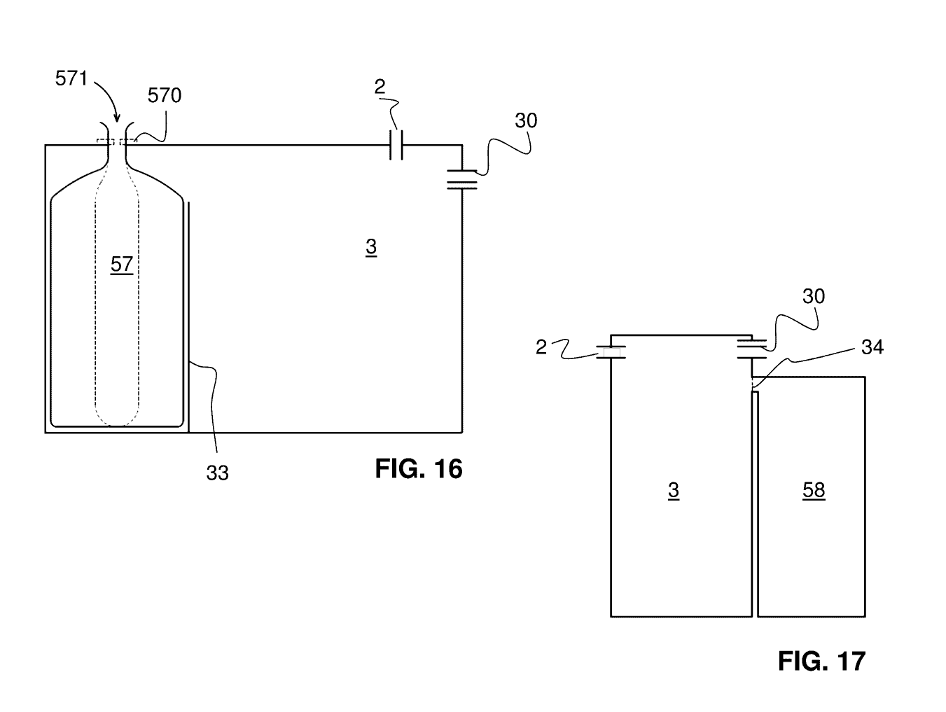

In the embodiments according to FIGS. 16 and 17, the system hardness of the drainage device is likewise generated by changing the container volume, but without the fluid collection container 3 being made partially flexible itself.

Arranged in the fluid collection container 3 according to FIG. 16 is a flexible insert container 57. The latter can be a bag, for example. This insert container 57 is connected to the outside of the fluid collection container 3 via a filling opening 571. The filling opening 571 can be closed with a closure piece 570. A non-compressible fluid, e.g. water, can be introduced in a predetermined quantity into the insert container 57, such that the insert container 57 occupies a predefined volume inside the fluid collection container 3. In this way, the air volume of the fluid collection container 3 available for the pressure equalization with the pleural cavity becomes smaller and the system becomes harder. With improved healing, the insert container 57 is filled to a greater extent in order to prepare the lung for the completion of the drainage.

In the embodiment according to FIG. 16, the fluid collection container 3 has an inner partition wall 33, which delimits the insert container 57 from the rest of the interior. Air exchange is therefore still possible between the subregions in the inside of the fluid collection container 3. This partition wall 33 is optional. The insert container 57 can also be arranged in another interior or in the sole interior of the fluid collection container 3.

In the embodiment according to FIG. 17, an attachment container 58 is present which is connected to the fluid collection container 3 via an air exchange opening 34. The attachment container 58 can preferably be plugged onto the fluid collection container 3 or otherwise secured thereto. The attachment container 58 can be stiff and rigid like the fluid collection container 3. However, it is preferably flexible such that its volume adapts at least partially to the underpressure prevailing in the interior of the containers. An attachment container of this kind is present at the start of the drainage. It can be replaced by a smaller attachment container as the drainage proceeds. At the end of the drainage, it is preferably only the fluid collection container 3 that is still in use, in which case the air exchange opening 34 is then closed in an airtight manner.

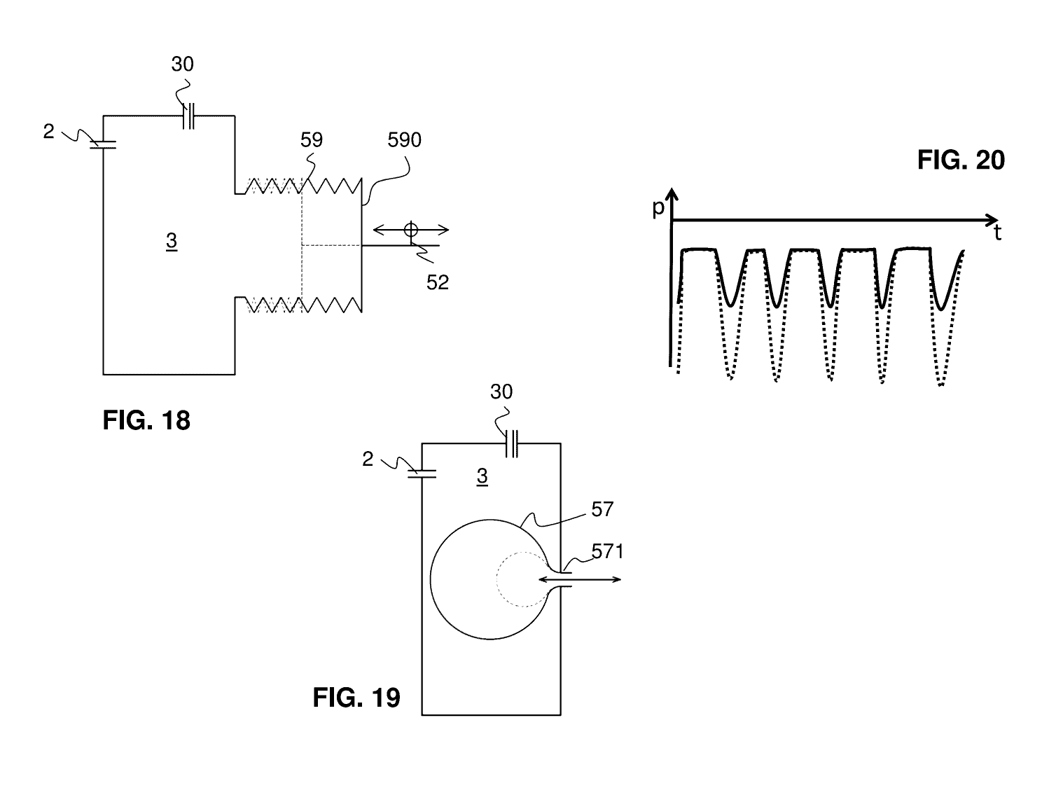

FIGS. 18 and 19 show embodiments that permit rapid, active regulation of the underpressure in the pleural cavity. A bellows 59 is arranged on the fluid collection container 3 according to FIG. 18, which bellows 59 is open to the interior of the fluid collection container 3 and closed to the environment. This bellows 59 has a stiff wall 590 which, by means of an anchor 52, can be moved towards the interior of the container 3 and away therefrom. In this way, the inner volume of the bellows 59 can change. The movement of the anchor 52 and of the bellows 59 can be effected manually, wherein the wall 590 is fixed at a different distance from the interior depending on the state of healing, and the hardness of the drainage system is thus adjusted. The closer the wall 590 to the fluid collection container 3 and the smaller the internal volume of the bellows 59, the harder the overall system.

Active regulation can be achieved by means of the anchor being connected to an electric motor and being moved via a control system. It can be brought to a fixed position depending on the healing process and can remain there for a period of several hours. However, the pressure is preferably monitored in the drainage tube or in a parallel auxiliary line connected thereto or in the fluid collection container. The sensor value obtained provides information concerning the pressure change. The anchor is moved in accordance with this monitored pressure change. That is to say, if the patient inhales too deeply, and if a pressure differential peak is expected, then the wall 590 is moved towards the container 3, and the bellows 59 decreases in size. Air is conveyed from the fluid collection container 3 to the pleural cavity P. This is indicated in FIG. 18 by dashed lines. In this way, the pressure difference peaks shown by dashed lines in FIG. 20 can be reduced or deliberately occasioned during inhalation, as can be seen by comparison with the more strongly attenuated pressure curve shown by solid lines.

The same result according to FIG. 20 can also be achieved with the embodiment according to FIG. 19. Here, a flexible insert container 57, in this case a balloon, is again arranged in the fluid collection container 3. The insert container 57 has an opening 571 directed to the outside, said opening 571 in this case being provided with a valve (not shown). Through this opening 571, air is blown and aspirated into the insertion container 57, preferably likewise in accordance with a measured pressure change, and preferably pneumatically. If inhalation is too deep, the air is blown in, and, upon normalization of the situation, i.e. when breathing is once more shallow, it is aspirated again. Pressure difference peaks can also be brought about by choosing the aspirated or blown in quantity of air.

An insert container 57 is not absolutely necessary. The air can also be blown directly into the fluid collection container 3 or aspirated directly therefrom.

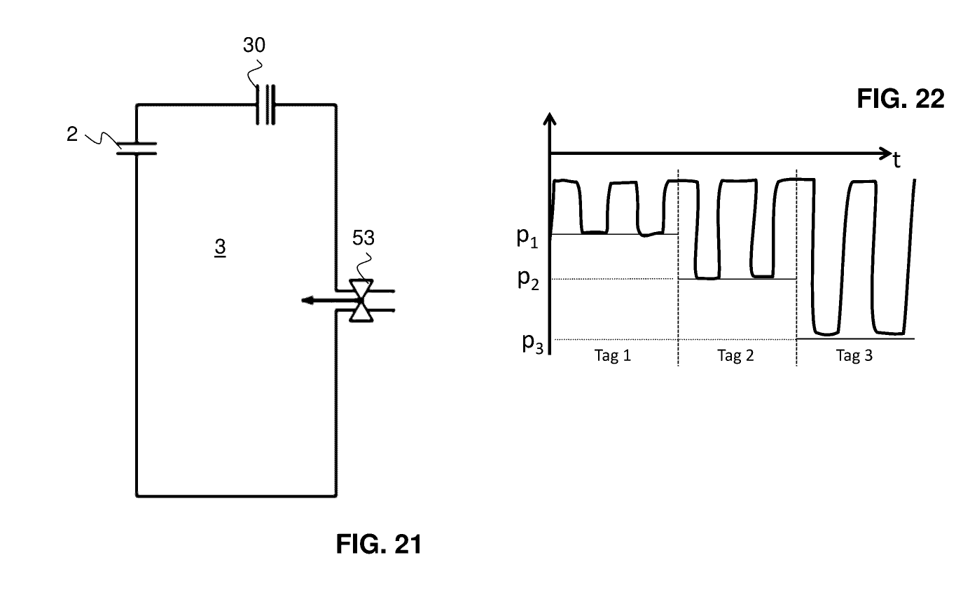

In the embodiment according to FIG. 21, a manually or electronically actuated valve 53 is present which opens at a predefined limit pressure in the interior of the fluid collection container 3. In this way, air can flow from outside into the fluid collection container 3 and reduce the pressure difference with respect to the atmosphere. As healing of the lung progresses, the limit pressure is set differently, such that the valve 53 opens only at a greater pressure difference. For example, the valve 53 can open on the first day at an underpressure of −2 kPa in the container 3, on the second day at −4 kPa, and on the third day at −6 kPa. FIG. 22 shows the pressure profile in the pleural cavity during inhalation and exhalation, which reflects the result of such valve adjustment.

The above-described static embodiments according to FIGS. 11 to 15 and FIG. 21 can likewise be analogously actuated automatically. They can also be provided with a control system in order to achieve, according to measured pressure values, an automatic active regulation of the hardness of the drainage system, so as to obtain the result according to FIG. 20.

This active regulation is not only advantageous in preparation for completion of the thorax drainage. It also serves to generally prevent abrupt peaks when inhalation is inadvertently too deep and to avoid the risk of an unexpected interruption in the drainage, e.g. upon clamping of the drainage tube or upon inadvertent interruption of the suction pump. If there is a threat of a pressure difference peak during inhalation, the overall system is made softer in order to smooth the pressure difference peak in the pleural cavity and to prevent excessive expansion of the lung.

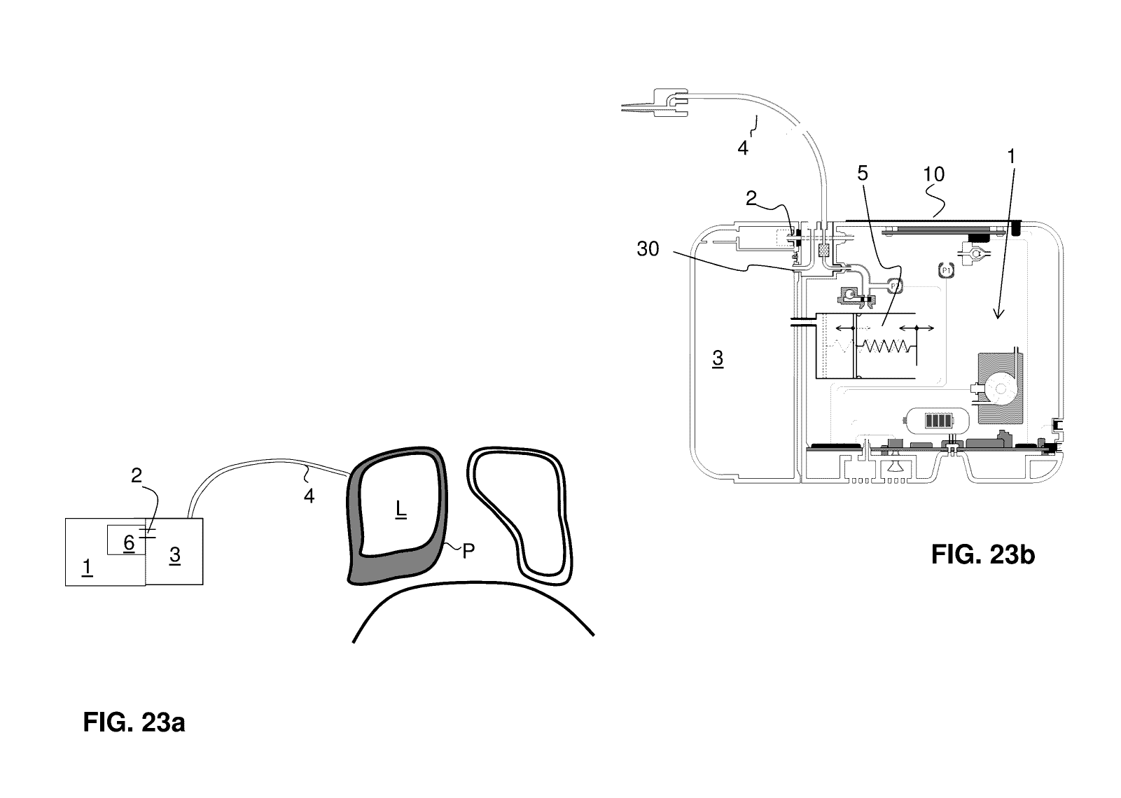

The examples described above concern changes in or on the fluid collection container 3. These changes can also occur in the housing of the suction pump 1. That is to say, pressure compensation containers 6 or valves, for example, can be arranged on the suction tube 2 or on the vacuum attachment of the suction pump 1, wherein the pressure compensation container 6 can be provided with the above-described membranes, insert containers or bellows. This is shown in FIG. 23a. Moreover, such mechanisms 5 for attenuating pressure differences can also be arranged in a housing 10 of the suction pump 1, wherein the connection to the fluid collection container 3 is effected via the suction opening or, as is shown in FIG. 23b, via an additional opening of the fluid collection container 3. Moreover, there are also other possible arrangements. Identical parts are designated in FIG. 23b using the same reference numbers as before.

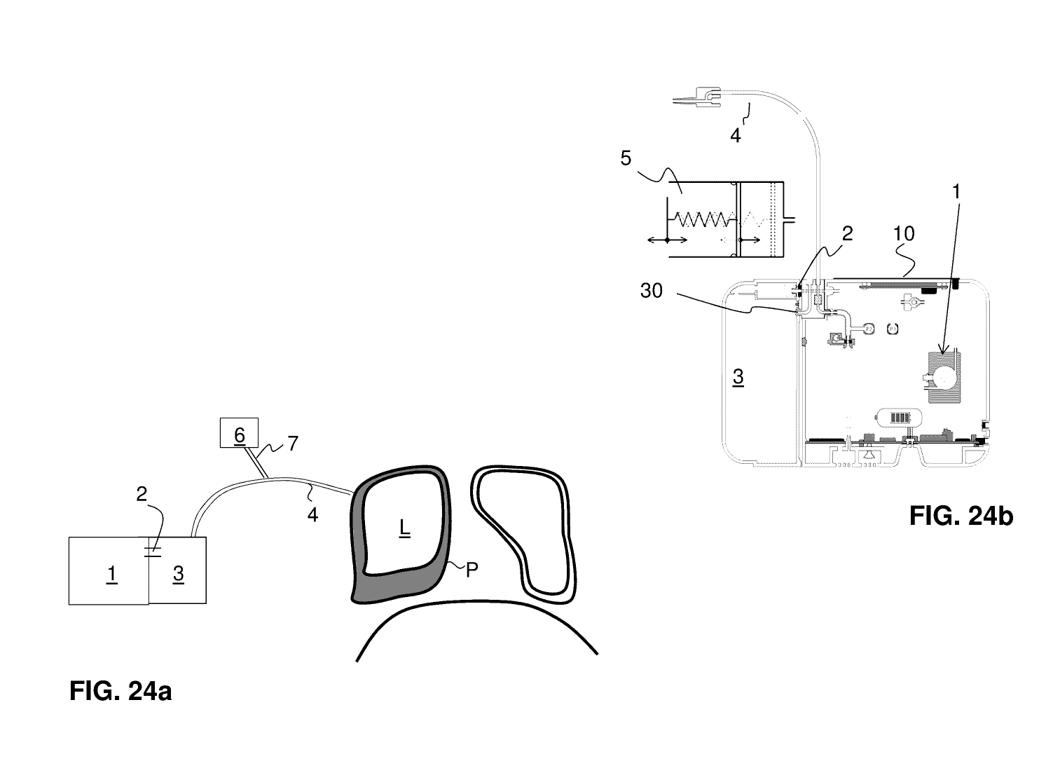

The drainage tube 4 can likewise be provided with a branch line 7, which leads to a compensation container 6 of this kind or a valve. This is shown in FIG. 24a. A specific arrangement in a housing 10 of a pump 1 is shown in FIG. 24b, where the attenuating mechanism 5 is shown but not a housing 6 surrounding it. Identical parts are shown by way of identical reference numerals.

The examples described here also function with regulated suction pumps, which monitor and regulate the underpressure in the drainage system. For example, this underpressure can be monitored in the pleural cavity, in the drainage tube, in an auxiliary line or in the fluid collection container. The reason for this is that the regulation performed by means of the suction pump is too slow to compensate for the pressure changes between inhalation and exhalation. However, the adjustable hardness of the system according to the invention permits static and dynamic compensations, which are fast enough to train the lung in such a way that there are no abrupt pressure differences upon completion of the drainage, and the lung is thereby protected.

The system according to the invention prevents an abrupt expansion of the lung and thus permits optimal training of the lung for the time when thorax drainage is completed.

What is claimed is:

1. A thorax drainage device for aspirating fluids from a pleural cavity of a patient by means of underpressure,
the thorax drainage device comprising a fluid collection container for collecting the aspirated fluids and a drainage tube for connecting the fluid collection container to the pleural cavity of the patient,
wherein the fluid collection container is connectable to a vacuum source, in order to generate an underpressure in the fluid collection container,
and wherein the thorax drainage device has an adjustable mechanism for attenuating pressure differences during the respiration of the patient, this mechanism being adjustable independently of a suction capacity of the vacuum source.

2. The thorax drainage device according to claim 1, wherein the mechanism for attenuating pressure differences is a mechanism for adjusting a return flow of air to the pleural space.

3. The thorax drainage device according to claim 2, wherein the mechanism for adjusting the return flow of air is adjustable manually or automatically.

4. The thorax drainage device according to claim 2, wherein the mechanism for adjusting the return flow of air is adjustable automatically and regulates the adjustment according to a sensor value.

5. The thorax drainage device according to claim 1, wherein the mechanism for attenuating pressure differences is arranged one of (a) between secretion collection container and suction source, (b) in the housing of the suction source, (c) in or on the secretion collection container, or (d) in or on the drainage tube.

6. The thorax drainage device according to claim 1, wherein the mechanism for attenuating pressure differences has a chamber, of which the stiffness is adjustable.

7. The thorax drainage device according to claim 1, wherein the mechanism for attenuating pressure differences has a chamber with an interior and with an opening that leads to the patient, wherein the chamber is formed by stiff walls, with the exception of a flexible membrane let into a wall of the chamber, and wherein the flexibility of the membrane is adjustable.

8. The thorax Thorax drainage device according to claim 7, wherein the membrane is spring-loaded.

9. The thorax drainage device according to claim 1, wherein the mechanism for attenuating pressure differences has a chamber with an interior and with an opening that leads to the patient, wherein the chamber is formed by stiff walls, with the exception of a spring-loaded piston which forms part of a wall, and of which the position relative to the interior is adjustable.

10. The thorax drainage device according to claim 1, wherein the mechanism for attenuating pressure differences has a chamber with an interior and with an opening that leads to the patient, wherein the chamber is formed by stiff walls, and wherein an insert container is arranged in the chamber, which insert container can be filled from the outside with a non-compressible fluid in order to limit the volume of the interior adjustably.

11. The thorax drainage device according to claim 1, wherein the mechanism for attenuating pressure differences has a chamber with an interior and with an opening that leads to the patient, wherein the chamber is formed by stiff walls, with the exception of a flexible bellows which forms part of a wall and which has an interior open towards the interior of the chamber, wherein the volume of the interior of the bellows is adjustable.

12. The thorax drainage device according to claim 1, wherein the mechanism for attenuating pressure differences has a first chamber with an interior and with an opening that leads to the patient, wherein the first chamber is formed by stiff walls, wherein one wall has a closable first air exchange opening for connection to a second chamber, which is closed except for a second air exchange opening, wherein the first chamber can be connected to the second chamber for air communication via the two air exchange openings.

13. The thorax drainage device according to claim 1, wherein the mechanism for attenuating pressure differences has a chamber with an interior and with an opening that leads to the patient, wherein the chamber is formed by stiff walls, and wherein the chamber has a filling opening, which is independent of any suction opening connected to the suction source and through which air can be blown or pumped into the chamber for the purpose of adjusting the attenuation of the respiration.

14. The thorax drainage device according to claim 1, wherein the mechanism for attenuating pressure differences has a chamber with an interior and with an opening that leads to the patient, wherein the chamber is formed by stiff walls, and wherein the chamber has a valve which leads to the outside and which opens outwards according to a detected underpressure.

15. The thorax drainage device according to claim 7, wherein this chamber (a) is formed by the fluid collection container, (b) is arranged in or on the fluid collection container, (c) is connected by a branch line to the drainage tube, or (d) is arranged between suction source and fluid collection container.

16. The chamber for use in a thorax drainage device for aspirating fluids from a pleural cavity of a patient by means of underpressure, the thorax drainage device comprising:
a fluid collection container for collecting the aspirated fluids; and
a drainage tube for connecting the fluid collection container to the pleural cavity of the patient,
wherein the fluid collection container is connectable to a vacuum source, in order to generate an underpressure in the fluid collection container,
wherein the thorax drainage device has an adjustable mechanism for attenuating pressure differences during the respiration of the patient, this mechanism being adjustable independently of a suction capacity of the vacuum source, and wherein the chamber has an adjustable stiffness and is adapted to form a part of the mechanism for attenuating pressure differences of the thorax drainage device.

17. A method for thorax drainage, wherein fluid from a pleural cavity of a patient is aspirated by means of underpressure into a fluid collection container, wherein the method entails the gradual or regulated adjustment of the attenuation of pressure differences during the respiration of the patient.
